(19) United States
(12) Patent Application Publication
(10) Pub. No.: US 2003/0069200 A1
(43) Pub. Date: Apr. 10, 2003

(54) Use of glucosylceramide synthesis inhibitors in brain cancertherapy

(76) Inventors: Frances Mary Platt, Oxford (GB); Thomas Neil Seyfried, Boston, MA (US); Paul David Smith, Abingdon (GB)

Correspondence Address: David A. Jackson, KLAUBER & JACKSON, 4th Floor, 411 Hackensack Street, Hackensack, NJ, 07601, US

(21) Appl. No.: 10/244,047

(22) Filed: Sep. 13, 2002

Related U.S. Application Data
(63) Continuation of application No. PCT/GB01/01189, filed on Mar. 19, 2001.

(30) Foreign Application Priority Data
Mar. 17, 2000 (GB) 0006539.1

(51) Int. Cl. A61K 48/00; A61K 31/45

(52) U.S. Cl. 514/44; 514/328

(57) ABSTRACT

Treatments for brain cancer are provided, based on administration of inhibitors of glycophospholipids synthesis.

The present invention provides the use of inhibitors of glycolipid synthesis in the manufacture of medicaments for use in the treatment of brain cancer.

Chemotherapy has generally been less effective for the treatment of brain cancers than for the treatment of non-brain cancers. This has been due in part to difficulties in penetrating the blood brain barrier and to the general inaccessibility of tumour cells after invading the neural parenchyma. The highly invasive properties of most malignant brain tumours protects them from direct chemical, radiological, or surgical assault (Harbaugh et al. 1998. Semin-Surg-Oncol. 14:26-33). Recent strategies for chemotherapy have focused on small molecules that specifically block growth-factor receptors (Barinaga 1997. Science 278:1036-1039). The rationale for these strategies comes from findings that many cancers, including gliomas, involve quantitative or qualitative abnormalities in various growth factor receptors, e.g. epidermal growth factor (EGF), platelet derived growth factor (PDGF), and basic fibroblast growth factor (bFGF) (Barinaga 1997. Science 278:1036-1039).

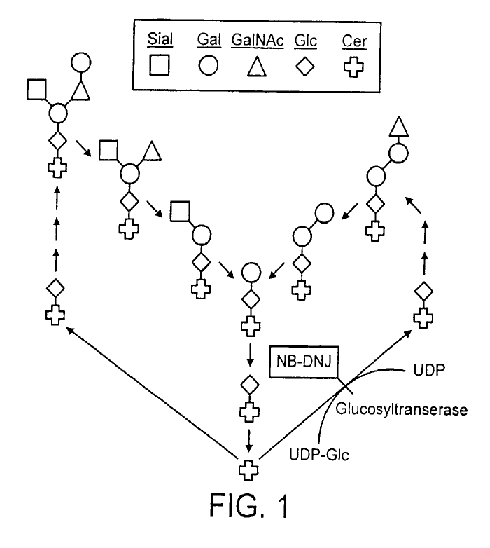

The glycophospholipids comprise the gangliosides and the neutral glycosphingolipids (GSLs), and are anchored in the outer surface of plasma membranes through their lipophilic ceramide tail. The GSL oligosaccharide head groups can interact with numerous cell surface receptors and can also facilitate cell signalling cascades through the ceramide (Bai et al. 1997. J. Lipid Res. 38:160-172, Ariga et al. 1998. J. Lipid Res. 39:1-16). The gangliosides are distinguished from the neutral GSLs in having sialic acid (N-acetylneuraminic acid) as part of the oligosaccharide chain (see FIG. 1 of the accompanying drawings). Mammalian gangliosides are synthesised by the stepwise addition of sugar residues to the oligosaccharide head group. This is accomplished through the action of a Golgi-bound multi-glycosyltransferase system, where the GSL product of one transferase serves as the substrate for another transferase (Sandhoff et al. 1994. Prog. Brain Res. 101:17-29, Seyfried et al. 1994 J. Lipid Res. 35:993-10016). Defects in ganglioside biosynthesis are found in most human cancers and are thought to underlie the invasive and malignant properties of brain tumours (Hakomori 1996. Cancer Res. 56:5309-5318, Fredman et al. 1996 Glycoconj. J. 13:391-399).

In vitro studies of D-threo-1-phenyl-2-decanoylamino-3-morpholino-1-propanol (D-PDMP), which is known to have some glucosylceramide synthesis inhibition activity, have shown that it reduces ganglioside shedding that masks immune recognition from blocking the growth of neuroblastoma tumours (Li et al (1996) Cancer Res 56:4602-5). Similarly, PDMP has been shown to inhibit neurite outgrowth by neuroblastoma cells in vitro (Uemura et al (1990) J Biochem (Tokyo) 110:96-102). However, PDMP is known to inhibit sphingomyelin synthesis, growth factor signalling, and protein and membrane transport (Rosenwald et al, (1992) Biochemistry 31:3581-90; Mutoh et al, (1998) J Biol Chem 273:26001-7; Kok et al, (1998) J Cell Biol 142:25-38; Rosenwald et al (1992) Biochemistry 31:3581-90). Therefore, the data provided by Li and Uemura makes it impossible to predict whether the effects of PDMP observed are caused by the inhibition of glucosylceramide synthesis.

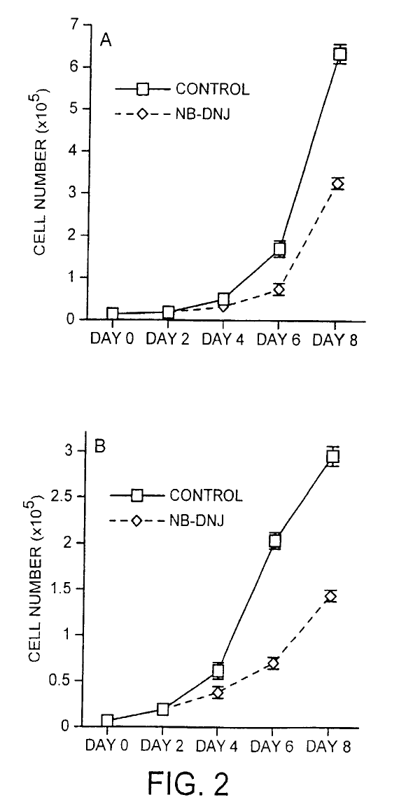

NB-DNJ inhibits the ceramide-specific glucosyltransferase (GlcT-1) that catalyses the first step in GSL biosynthesis (see FIGS. 1 and 2 of the accompanying drawings). Since GlcCer is the common metabolic precursor required for the synthesis of most gangliosides and neutral glycolipids, NB-DNJ treatment significantly reduces the content of all GSLs containing the GlcCer core structure.

We have now found that NB-DNJ inhibits the growth and ganglioside content of 20-methylcholanthrene-induced mouse brain tumours whether they were grown subcutaneously or intracranially. These tumours, which include the EPEN and CT-2A, were previously classified as poorly differentiated malignant astrocytomas and share a number of morphological and histological features with malignant human brain tumours (Zimmerman et al. 1941. Cancer Res. 1:919-938, Seyfriedet al. 1992. Mol. Chem. Neuropathol. 17:147-167). Furthermore, the reduced subcutaneous tumour growth correlated with reduced tumour ganglioside content.

Thus, in a first aspect, the present invention provides the use of an inhibitor of glucosylceramide synthesis, in the manufacture of a medicament for use in the treatment of brain cancer.

As used herein, “brain cancer” is intended to include primary brain tumours such as cancers of neuronal and glial origin, e.g. glioblastoma and astrocytoma, as well as secondary brain tumours which metastasise to brain tissue from non-brain tissue.

“Inhibitors” in accordance with the present invention are preferably specific inhibitors of glucosylceramide synthesis, that is to say that, although they may have other activities, which may be inhibitory, the predominant activity of the inhibitor is to inhibit glucosylceramide synthesis. Thus, the use of D-threo-1-phenyl-2-decanoylamino-3-morpholino-1-propanol in the manufacture of a medicament for the treatment of brain cancer is not included within the scope of the present application.

In the context of the present invention, the term “inhibitor” includes, but is not limited to, molecules such as N-butyldeoxynojirimycin, N-butyldeoxygalactonojirimycin and other imino sugar-structured inhibitors of glucosylceramide synthesis. Furthermore, inhibition can also be achieved by the use of genetic approaches, based on the introduction of nucleic acid coding for proteins or peptides capable of inhibiting glucosylceramide synthesis or antisense sequences or catalytic RNA capable of interfering with the expression of enzymes responsible for glucosylceramide synthesis (e.g. glucosylceramide synthase). A combination of any of the above inhibitors can be used.

In a second aspect, the present invention provides the use of N-butyldeoxynojirimycin in the manufacture of a medicament for use in the treatment of brain cancer.

In a third aspect, the present invention provides the use of an agent capable of increasing the rate of neuronal glycolipid degradation in the manufacture of a medicament for use in the treatment of brain cancer. Examples of such agents include enzymes which degrade neuronal glycolipids, e.g. lysosomal hexoseaminidases, galactosidases, sialidases and glucosylceramide glucosidase and molecules which increase the activity of such enzymes. In addition, the agent could comprise a nucleic acid sequence (DNA or RNA) which codes for the enzymes mentioned above, i.e. such sequences could be introduced to increase natural production of such enzymes.

Methods and processes for the production of N-butyldeoxynojirimycin can be found for example in U.S. Pat. No. 4,182,767, EP-B-0012278, EP-A-0624652, U.S. Pat. Nos. 4,266,025, 4,405,714 and 5,151,519 for example.

In other aspects, the present invention provides:

(a) a method for the treatment of brain cancer which comprises administering to a subject in need thereof a therapeutically effective amount of a glucosylceramide synthesis inhibitor;

(b) a method for the treatment of brain cancer which comprises administering to a subject in need thereof a therapeutically effective amount of N-butyldeoxynojirimycin;

(c) a method for the treatment of brain cancer which comprises administering to a subject in need thereof a therapeutically effective amount of an agent capable of increasing the rate of degradation of neuronal glycolipids.

The medicaments described herein and which are also for use in the methods provided herein, may include one or more of the following: preserving agents, solubilising agents, stabilising agents, wetting agents, emulsifiers, sweeteners, colorants, odourants, salts, buffers, coating agents or antioxidants. They may also contain therapeutically active agents in addition to the compounds and/or agents described herein.

Routes of Administration

The medicaments may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such a composition may be prepared by any method known in the art of pharmacy, for example by admixing the active ingredient with a carrier under sterile conditions.

Various routes of administration will now be considered in greater detail:

(i) Oral Administration

Medicaments adapted for oral administration may be provided as capsules or tablets; as powders or granules; as solutions, syrups or suspensions (in aqueous or non-aqueous liquids); as edible foams or whips; or as emulsions.

Tablets or hard gelatine capsules may comprise lactose, maize starch or derivatives thereof, stearic acid or salts thereof.

Soft gelatine capsules may comprise vegetable oils, waxes, fats, semi-solid, or liquid polyols etc.

Solutions and syrups may comprise water, polyols and sugars. For the preparation of suspensions oils (e.g. vegetable oils) may be used to provide oil-in-water or water-in-oil suspensions.

(ii) Transdermal Administration

Medicaments adapted for transdermal administration may be provided as discrete patches intended to remain in intimate contact with the epidermis of the recipient for a prolonged period of time. For example, the active ingredient may be delivered from the patch by iontophoresis (Iontophoresis is described in Pharmaceutical Research, 3(6):318 (1986)).

(iii) Topical Administration

Medicaments adapted for topical administration may be provided as ointments, creams, suspensions, lotions, powders, solutions, pastes, gels, sprays, aerosols or oils.

For infections of the eye or other external tissues, for example mouth and skin, a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base.

Medicaments adapted for topical administration to the eye include eye drops. Here the active ingredient can be dissolved or suspended in a suitable carrier, e.g. in an aqueous solvent.

Medicaments adapted for topical administration in the mouth include lozenges, pastilles and mouthwashes.

(iv) Rectal Administration

Medicaments adapted for rectal administration may be provided as suppositories or enemas.

(v) Nasal Administration

Medicaments adapted for nasal administration which use solid carriers include a coarse powder (e.g. having a particle size in the range of 20 to 500 microns). This can be administered in the manner in which snuff is taken, i.e. by rapid inhalation through the nose from a container of powder held close to the nose.

Compositions adopted for nasal administration which use liquid carriers include nasal sprays or nasal drops. These may comprise aqueous or oil solutions of the active ingredient.

Medicaments adapted for administration by inhalation include fine particle dusts or mists, which may be generated by means of various types of apparatus, e.g. pressurised aerosols, nebulisers or insufflators. Such apparatus can be constructed so as to provide predetermined dosages of the active ingredient.

(vi) Vaginal Administration

Medicaments adapted for vaginal administration may be provided as pessaries, tampons, creams, gels, pastes, foams or spray formulations.

(vii) Parenteral Administration

Medicaments adapted for parenteral administration include aqueous and non-aqueous sterile injectable solutions or suspensions. These may contain antioxidants, buffers, bacteriostats and solutes which render the compositions substantially isotonic with the blood of an intended recipient. Other components which may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

Dosages

Dosages will be readily determinable by routine trials, and will be under the control of the physician or clinician. The guiding principle for determining a suitable dose will be delivery of a suitably efficacious but non-toxic, or acceptably toxic, amount of material. A daily dosage for an adult could be expected to be in the range of from 10 to 6000 mg of active agent, preferably 100 to 4000 mg, more preferably 150 to 3000 mg. Doses of about 3000 mg, 150-300 mg or 600 mg may be used.

Preferred features of each aspect of the invention are as for each of the other aspects mutatis mutandis.

In the accompanying drawings:

FIG. 1 is a schematic representation of the synthesis and degradation of glucosylceramide-containing glycolipids. The enzyme reaction inhibited by N-butyldeoxynojirimycin to decrease the synthesis of glucosylceramide-containing glycolipids is also shown.

FIGS. 2A and 2B are graphs showing the number of CT-2A cells and EPEN cells respectively over time with (NB-DNJ) and without (control) the addition of NB-DNJ.

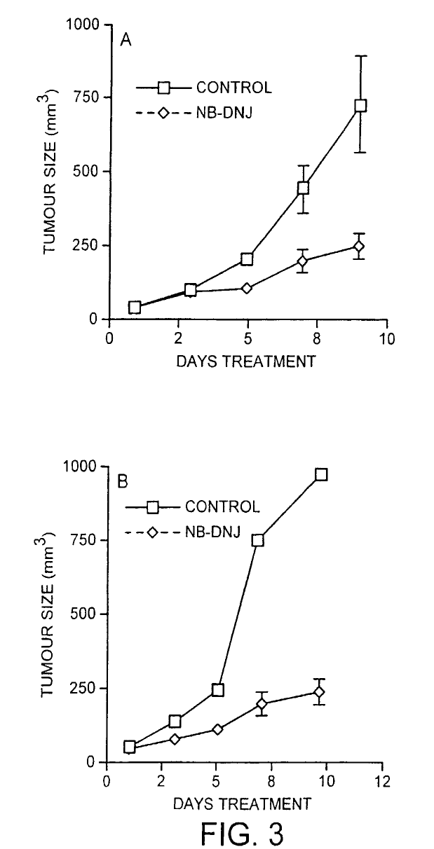

FIGS. 3A and 3B are graphs showing the tumour size for CT-2A and EPEN tumours respectively with (NB-DNJ) and without (control) the addition of NB-DNJ.

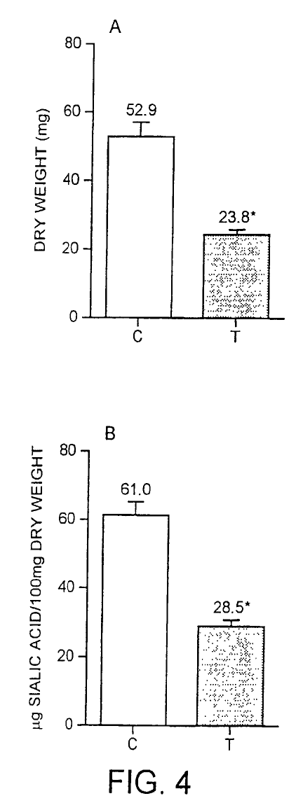

FIG. 4A is a graph comparing the dry weight of CT-2A brain tumours with (T) and without (C) the addition of NB-DNJ and FIG. 4B is a graph comparing the ganglioside content of CT-2A brain tumours with (T) and without (C) the addition of NB-DNJ.

The invention will now be described with reference to the following examples, which should not in any way be construed as limiting the scope of the invention.

### EXAMPLES

### Example 1

### NB-DNJ Inhibits Growth of Cultured Brain Tumour Cell Lines

Approximately 0.5×10⁴ cells for CT-2A and 1×10⁴ cells for EPEN were seeded in wells of a 24 well plate and cultured for 24 hours in DMEM supplemented with 10% FBS. The exact cellular origin of the CT-2A and EPEN tumours is not known, but Dr. Allan Yates, Chief of Neuropathology at Ohio State University classified the tumours as poorly differentiated anaplastic astrocytomas (see Seyfried et al Mol. Chem. Neuropathol. 17: 147-167, 1992).

Cells were cultured for a further 7 days in the same medium for control cells or in the presence of NB-DNJ (200 μM) for treated cells. Cells were counted on days 2, 4, 6 and 8 using a Coulter counter and the results are shown in FIGS. 2A and 2B where values are expressed as means±SEM (N=3).

The results show that NB-DNJ (200 μM) significantly reduced the growth of the CT-2A and the EPEN cultured brain tumour cell lines. Furthermore, the growth inhibitory effect was similar (about 50% inhibition after 8 days) for the rapidly dividing CT-2A tumour cells and the less rapidly dividing EPEN tumour cells. The percentage of dead cells did not differ significantly between the control and treated cultures (as detected by trypan blue exclusion). These findings suggest that NB-DNJ inhibits brain tumour growth by reducing tumour cell proliferation.

### Example 2

### NB-DNJ Inhibits Brain Tumour Growth in Mice

CT-2A and EPEN tumours were grown subcutaneously in the flanks of six-week old syngeneic C57BL/6 mice. The treated mice were maintained on a powdered mouse chow diet (Prolab, Agway) that contained NB-DNJ (2400 mg/kg body weight/day). The control mice received only the powdered mouse chow. Tumours were enzymatically dissociated as described in Ecsedy et al, J. Lipid Res., 39: 2218-2227, 1998 and approximately 5×10⁶ cells from each tumour were inoculated subcutaneously into the flanks of the host mice. For the CT-2A-bearing mice, drug treatment was started six days post-tumour cell inoculation and was continued for a total of nine days. For the EPEN-bearing mice, drug treatment was started eleven days post-tumour inoculation and was continued for a total of ten days. This was done because the EPEN tumour grows more slowly than the CT-2A tumour (Cotterchio et al. 1993. Mol. Chem. Neuropathol. 20:163-172). The method of Looney was used to determine tumour growth rates (volume increase/day) as previously described (Cotterchio et al. 1993. Mol. Chem. Neuropathol. 20:163-172). The results are shown in FIGS. 3A and 3B where values are expressed as means±SEM except for the control EPEN, which is the mean of two independent samples.

The results show that NB-DNJ (2400 mg/kg body wt) reduced the subcutaneous growth of the CT-2A and EPEN brain tumours. NB-DNJ reduced the growth rate of CT-2A tumour by 68% compared to the untreated controls and reduced that of the EPEN by 76%.

### Example 3

### NB-DNJ Inhibits Intra-Cranial Tumour Growth and Reduces Brain Tumour Ganglioside Content

CT-2A tumour tissue was minced and tissue pieces (about 1 mm³) were implanted into the cerebral cortex of C57BL/6J mice using a trocar as described previously (Seyfried et al. 1987. Cancer Res. 47:3538-3542). The NB-DNJ diet as in Example 2. Control mice received only the powdered mouse chow. Tumours were excised from surrounding brain tissue, frozen, and lyophilized to remove water. Gangliosides were isolated and purified and content expressed as μg sialic acid/100 mg dry weight, as determined using the resorcinol reagent, as previously described (Seyfried et al. 1978. J. Neurochem. 31:21-27). The results are shown in FIGS. 4A and 4B, in which values are expressed as means±SEM and n=the number of independent brain tumours analysed. * indicates that the dry weight or ganglioside content of the treated tumours was significantly lower than that of the control tumours at P<0.01 by two tailed Student's t-test.

The results show that NB-DNJ dietary treatment significantly reduced the intra-cranial growth of the CT-2A tumour and that NB-DNJ can penetrate the blood brain barrier. Since CT-2A is a rapidly growing malignant tumour (Cotterchio et al. 1993. Mol. Chem. Neuropathol. 20:163-172), these data also suggest that NB-DNJ may have therapeutic potential for human brain tumours.

In addition, NB-DNJ reduced ganglioside content in the CT-2A mouse brain tumour grown subcutaneously (See FIG. 4B and Table 1 below). The reductions were observed for gangliosides containing both N-acetylneuraminic acid (NeuAc) and N-glycolylneuraminic acid (NeuGc).

The reduction in ganglioside concentration (53%) intra-cranial tumours was strongly correlated with the reduction in tumour dry weight (55%). These findings suggest that NB-DNJ can penetrate the blood brain barrier and the neural parenchyma, and suggest that NB-DNJ may inhibit brain tumour growth through an effect on GSL biosynthesis.

TABLE 1
Influence of NB-DNJ on the Ganglioside Content of the CT-2A
Experimental Mouse Brain Tumour^{a}

Ganglioside Neuraminic Acid Content^{d}

| | (μg/100 mg dry wt) | | | |
|---|---|---|---|---|
| Tumour^{b} | N^{c} | Total | NeuAc | NeuGc |
| Control | 3 | 51.1 ± 7.7 | 26.0 ± 7.4 | 16.0 ± 0.8 |
| Treated | 2 | 27.1 | 21.8 | 5.6 |
| | | (30.6, 23.6) | (25.5, 18.1) | (5.6, 5.5) |

We claim:

1. A method for the treatment of brain cancer comprising administering to a subject in need thereof a therapeutically effective amount of a glucosylceramide synthesis inhibitor.

2. The method as claimed in claim 1, wherein the inhibitor is one or more imino sugar-structured inhibitors of glucosylceramide synthesis.

3. The method as claimed in claim 2, wherein the inhibitor comprises one or both of N-butyldeoxynojirimycin and N-butyldeoxygalactonojirimycin.

4. The method as claimed in claim 1, wherein the inhibitor comprises one or more of a nucleic acid coding for a protein or peptide capable of inhibiting glucosylceramide synthesis, and an antisense sequence or catalytic RNA capable of interfering with the expression of enzymes responsible for glucosylceramide synthesis.

5. A method for the treatment of brain cancer comprising administering to a subject in need thereof a therapeutically effective amount of N-butyldeoxynojirimycin.

6. A method for the treatment of brain cancer comprising administering to a subject in need thereof a therapeutically effective amount of N-butyldeoxygalactonojirimycin.

7. A method for the treatment of brain cancer comprising administering to a subject in need thereof a therapeutically effective amount of an agent capable of increasing the rate of neuronal glycolipid degradation.

8. The method as claimed in claim 7, wherein the agent comprises one or more of an enzyme which degrades neuronal glycolipids, a molecule which increases the activity of such an enzyme, and a nucleic acid sequence (DNA or RNA) which codes for such an enzyme.

9. The method as claimed in claim 8, wherein the enzyme is selected from a lysosomal hexoseaminidase, galactosidase, sialidase or glucosylceramide glucosidase.

10. The method as claimed in claim 1, wherein the brain cancer is a cancer of neuronal or glial origin, or a secondary brain tumour which has metastasised to brain tissue from non-brain tissue.

11. The method as claimed in claim 5, wherein the brain cancer is a cancer of neuronal or glial origin, or a secondary brain tumour which has metastasised to brain tissue from non-brain tissue.

12. The method as claimed in claim 6, wherein the brain cancer is a cancer of neuronal or glial origin, or a secondary brain tumour which has metastasised to brain tissue from non-brain tissue.

13. The method as claimed in claim 7, wherein the brain cancer is a cancer of neuronal or glial origin, or a secondary brain tumour which has metastasised to brain tissue from non-brain tissue.
